(19) United States
(12) Patent Application Publication
(10) Pub. No.: US 2009/0068296 A1
(43) Pub. Date: Mar. 12, 2009

(54) COMPOSITION FOR IMPROVED PRODUCTION, HEALTH AND FERTILITY TRAITS

(76) Inventor: Douglas L. Greger, Plainview, NY (US)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/280,522

(22) PCT Filed: Feb. 22, 2007
(86) PCT No.: PCT/US07/04453

§ 371 Date: Aug. 22, 2008

(30) Foreign Application Priority Data
Feb. 22, 2006 (US) 60775353

(51) Int. Cl. A61K 36/38; A61K 36/282; A61K 36/8962; A61K 36/16; A61K 36/00; A01K 67/027; A23L 1/31; A61K 36/67; A61K 36/752; A61K 36/28

(52) U.S. Cl. 424/730; 424/740; 424/754; 424/752; 424/736; 424/764; 424/734; 424/725; 426/641; 800/8

(57) ABSTRACT

This invention relates to products and methods for improving sensory qualities of meat obtained from livestock. In particular, this invention relates to products and methods for reducing off-odors and off-tastes in meat from uncastrated male pigs (boar taint) or from other livestock fed on pasture. According to the invention, this is obtained by administering a product that contains an inducer of the constitutive androstane receptor (CAR). These include plant products derived from Artemisia spp, Citrus spp., Gingko biloba, Tagetes lucida, as well as vitamin E (alpha tocopherol). The CAR-inducer products of the invention may be used to reduce boar taint and thereby eliminate the need for castration of male pigs.

### RELATED APPLICATIONS

The present application is based on International Application Number PCT/US2007/004453 filed Feb. 22, 2007, and claims priority from U.S. Application No. 60/775,353 filed Feb. 22, 2006, the disclosures of which are hereby incorporated by reference herein in their entirety.

### BACKGROUND

The present invention relates to the stimulation of CAR and/or PXR expression by feeding compositions containing one or more of these constituents such that improvements in several economically important traits may be realized.

### SUMMARY OF THE INVENTION

The present invention relates to feed compositions for mammals to be used for the purpose of improving production traits, which includes carcass composition, fat quality, energy homeostasis, and fertility. Specifically, this invention relates to the use of one or more herbal supplements that influence these traits by increasing the expression of constitutive androstane receptor (CAR) and/or pregnane X receptor (PXR). Herbal supplements that may be used include Artemisia species (wormwoods), such as Virgate Wormwood (Artemisia capillaris), Red Stem Wormwood (Artemisia scoparia), and other Artemisia species. Other plant or yeast products may also be used in the present formulation, which includes citrus (Citrus spp.) peel extracts as well as garlic (Allium sativum), St. John's Wort (Hypericum perforatum), kava (Piper methysticum), vitamin E (alpha-tocopherol) and extracts of Red Yeast (Monascus purpureus).

It is known that these botanicals and herbs affect metabolism by their influence on expression of nuclear receptors and nuclear receptor target genes in animal tissues, such as liver. Two nuclear receptors in particular that are affected by these compounds are constitutive androstane receptor (CAR) and pregnane X receptor (PXR). Artemisia spp. and Citrus peel contains scoparone, a compound known to induce CAR expression. In addition, garlic and Red Yeast extracts also increase expression of CAR. St. John's Wort and kava increase expression of PXR. The present invention relates to the stimulation of CAR and/or PXR expression by feeding compositions containing one or more of these or similar herbs such that improvements in several economically important traits may be realized. For example, we have shown that increased expression of CAR mRNA is related to increased expression of liver genes that control the metabolism of compounds that cause off-odors in pork. The inventor has shown that hepatic mRNA expression of CAR is highly correlated with mRNA expression of cytochrome P450 2E1 (CYP2E1), cytochrome P450 2A6 (CYP2A6), cytochrome P450 1A2 (CYP1A2), pregnane X receptor (PXR), UDP-glucoronysl transferase 1A1 (UGT 1A1). These genes are involved in fat metabolism as well as endobiotic and xenobiotic metabolism. The inventor has also shown that hepatic mRNA expression of CAR is highly correlated with the reduced presence of xenobiotic compounds in the fat, such as skatole and indole, that cause undesirable flavors and odors when present in high amounts in the meat or milk of pigs, cattle and sheep.

Thus, one aspect of the present invention is to feed an herbal supplement composed of one or more of the subject herbs such that CAR expression and/or activity is induced (CAR inducers) for the purpose of improving sensory qualities of meat. The subject herbal supplement may be fed to male pigs for the purpose of reducing the presence of compounds in fat that cause sensory defects, a phenomenon known as “boar taint”. Similarly, it is expected that induction of CAR could be used to improve sensory qualities of milk, as from a dairy cow.

Induction of CAR expression and activity may also be used to facilitate expression of enzymes involved in metabolism and excretion of potentially toxic foreign compounds (xenobiotics) as well as potentially toxic byproducts of metabolism (endobiotics) such as bilirubin. This may be particularly useful as a feed supplement for cats, which typically exhibit poor ability to conjugate and excrete such compounds.

Similarly, induction of CAR expression and activity by the use of herbal feed supplements may be utilized for the purpose of increasing ovulation rate (the number of eggs ovulated) and litter size in female animals, which includes domestic animals such as pigs, cattle, sheep, goats and poultry. Herbal CAR inducers may also be used to increase the likelihood of multiple ovulations in humans. Similarly, CAR inducers may be utilized for treating problems related to ovulation failure. In particular, CAR inducers may be utilized for the treatment or prevention of cystic ovarian follicles in female mammals; such herbal supplements may therefore also be used for treatment or prevention of Polycystic Ovarian Syndrome (PCOS) in humans, which is a common cause of infertility among women.

Herbal inducers of PXR may also be used as feed supplements, since PXR affects many of the same genes as does CAR. Thus, it is envisioned that herbal inducers of CAR and PXR may be utilized either alone or in combination for the purposes listed above. It is also envisioned that CAR and PXR inducers will be additive to or synergistic with other treatments that are used to improve the above-mentioned traits. For example, combined use of CAR and/or PXR inducers with compositions containing omega-3-fatty acids (fish oils) may be used together in order to achieve the maximum improvements in ovulation rate and/or litter sizes above the level that either treatment is able to attain when administered alone.

### DETAILED DESCRIPTION

The inventor has made the novel discovery that mRNA expression of Constitutive Androstane Receptor (CAR; NR1I3) in the tissues of mammals is related to expression of genes involved in metabolic processes that influence metabolism of nutritional byproducts that are known to cause sensory problems of odor and taste in meat. Specifically, the present discovery demonstrates that mRNA expression of CAR is highly correlated with mRNA expression of target genes important in fat metabolism as well as with genes involved in metabolism of endobiotics and xenobiotics. Endobiotics refer to compounds that are byproducts of normal metabolism that may be toxic if not transformed and eliminated. Xenobiotics refer to compounds derived from feed or other exogenous sources that also are potentially toxic to the animal.

For example, the inventor has shown that increased expression of CAR mRNA is related to increased expression of liver genes that control the metabolism of compounds that cause off-odors in pork. Thus, one aspect of the present invention is to feed a supplement composed of one or more of the subject herbs, extracts, or vitamins such that CAR expression and/or activity is induced (CAR inducers) for the purpose of improving sensory qualities of odor and taste of meat. The subject supplement may be fed to male pigs for the purpose of reducing the presence of compounds in fat that cause sensory defects of odor and taste, a phenomenon known as “boar taint”. It is also expected that induction of CAR expression could be used to improve sensory qualities of meat from other genders of pigs, such as castrates and females. Similarly, it is expected that induction of CAR could be used to improve sensory qualities of milk, as from a dairy cow.

Induction of CAR expression and activity may also be used to facilitate expression of enzymes involved in metabolism and excretion of potentially toxic foreign compounds (xenobiotics) as well as potentially toxic byproducts of metabolism (endobiotics) such as bilirubin. For example, CAR inducers may be used to mitigate the negative effects of toxic substances, such as mycotoxins on growth and reproductive traits in animals.

It is known that particular plants, plant extracts, and vitamins affect metabolism by their influence on expression of nuclear receptors and nuclear receptor target genes in animal tissues, such as liver. One nuclear receptor in particular that is affected by these compounds is constitutive androstane receptor (CAR). Artemisia spp., Tagetes lucida, and Citrus peel contains scoparone, a compound known to induce CAR expression. In addition, alpha-tocopherol also is known to increase expression of CAR. Gingko biloba has also been shown to increase expression of CAR target genes, such as those in the CYP2B family.

### Reduction to Practice:

Two pilot studies have been conducted to demonstrate proof of concept for the use of Artemisia capillaris, or other feed supplements containing the active component(s), to be fed to pigs for the reduction of boar taint compounds in pig fat. In the first study, entire male pigs (non-breeding boars, 5-6 months of age) were divided into three groups of fifteen boars each, comprising a control group and two treatment groups. The treatment groups were fed a dried aqueous extract of Artemisia. capillaris at two different inclusion rates (15 g/day or 60 g/day). After 8 days of treatment, pigs were transported and slaughtered at a plant by electrical stunning, and samples taken of kidney fat, back fat and liver. Fat samples were placed in plastic bags and kept on ice before being trimmed and stored in vacuum bags at −20 C. Fat samples were analyzed according to previously validated methods (Dehnhard, M., R. Claus, M. Hillenbrand, and A. Herzog, 1993: High performance liquid chromatographic (HPLC) method for the determination of 3-methylindole (skatole) and indole in adipose tissue of pigs. J. Chromatogr. 616, 205-209).

The results showed that feeding of Artemisia capillaris extract for a period of eight days prior to slaughter caused a reduction in the concentrations of skatole in back fat obtained from treated pigs. In the low-dose group, skatole levels were 50% less than controls, and in the high dose group skatole levels were 60% lower than controls. The reduction in skatole in both treatment groups compared with controls was statistically significant (P<0.05).

A second pilot study was conducted in order to evaluate whether the aqueous extract used was more efficacious than feeding the dried Artemisia capillaris. Entire male pigs (non-breeding boars, 5-6 months of age; 15 per group) were divided into a control and two treatment groups. The feed supplement treatments were either an aqueous extract of Artemisia capillaris at an inclusion level of 30 g/day, or dried Artemisia capillaris at an inclusion rate of 75 g/day. The results clearly indicated that feeding of the aqueous extract to boars for a period of eight days caused a reduction in the concentrations of skatole in the backfat at slaughter.

FIG. 1: Skatole concentrations in backfat of boars fed either aqueous extract or dried preparations of Artemisia capillaris compared with controls.

| | Extract | Dried A. capillaris | Controls |
|---|---|---|---|
| Skatole (μg/g fat) | 0.07 ± 0.01* | 0.11 ± 0.04 | 0.16 ± 0.03 |

*Mean ± standard error; extract versus controls, P < 0.02.

Importantly, the percentage of boars that exhibited skatole concentrations in the backfat that were higher than the generally accepted sensory threshold (≧0.20 μg/g fat) were reduced from 33% in the controls, 13.3% in the dried herb group, and to 0.0% in the aqueous extract group. The reduction in the frequency of boars with high skatole levels in the extract group compared with controls was statistically significant (P<0.05; Chi square analysis).

The use of the plant extracts and vitamins of the present invention for the reduction of off-odors and off-tastes associated with boar taint is novel and has not been reported or suggested in the prior art. Artemisia capillaris has been suggested for use as a feed ingredient for improving growth rates of pigs (PCT WO 2006/006768), and was suggested to improve meat quality as measured solely by increasing fat content of meat. However the application for reducing boar taint and eliminating the need for castration of male pigs has not been indicated in the prior art. Importantly, the present invention is novel in that the time period for feeding the compositions of the present invention are quite short, only from 2 to 21 days prior to slaughter.

Preferably the livestock feed of the present invention contains plant extracts obtained from Artemisia species, such as Artemisia capillaris, Artemisia scoparia, Artemisia annua, or from Gingko biloba, and or peel or leaf extracts from Citrus species. In a preferred embodiment, the plant extract includes an aqueous extract of Artemisia species or, Gingko biloba, or Citrus peel. Methods for preparation of aqueous plant extracts are well known in the prior art. It is also preferred to prepare a premix of plant extracts or vitamins, which may comprise an amount equivalent of 1% to 80% of such a premix. The premix may comprise other dietary supplements and may be mixed with conventional feed to obtain the feed of the invention, but this depends on the amount of premix to be used in the final feed composition. The amount of premix used is typically between 0.1 to 20% based on the total weight of the mixed feed. For the purposes of reducing boar taint, the livestock feed of the present invention is intended to be fed for a short time period prior to slaughter, such as from 2 to 21 days. It is preferred to feed the supplement for 4-7 days prior to slaughter.

1.-11. (canceled)

12. A composition to be administered to an animal comprising an active amount of one or more nuclear receptor inducers for the purpose of improving sensory qualities of odor or taste of meat or milk.

13. A composition to be administered to an animal comprising an active amount of one or more nuclear receptor inducers for the purpose of improving metabolism and excretion of potentially toxic compounds.

14. A composition to be administered to a female animal comprising an active amount of one or more nuclear receptor inducers for the purpose of increasing ovulation rate and/or litter size.

15. The composition of claim 12 wherein the nuclear receptor inducer is one that induces constitutive androstane receptor (CAR) or pregnane X receptor (PXR) including but not limited to preparations from one or more of Artemisia capillaris, Artemisia scoparia, Artemisa annua, other Artemisia spp., Allium sativa, Gingko biloba, Citrus spp, Tagetes lucida, Hypericum perforatum, Piper methysticum, or Monascus purpureus.

16. A method of improving the sensory qualities of odor or taste of meat or milk of an animal comprising administering orally, for example as a feed supplement, a composition as in claim 15.

17. The composition of claim 15 whereby the feed supplement is composed of one or more CAR inducers, or an extract thereof, in an amount equivalent to 0.1% to 20% of the total amount of livestock feed.

18. The method of claim 16 whereby the feed supplement is administered to a meat animal for 2 to 21 days prior to slaughter.

19. The method of claim 16 whereby the feed supplement is administered to a meat animal for 4-7 days prior to slaughter.

20. A method of claim 16 where the meat animal is an uncastrated male pig.

21. The composition of claim 13 whereby the nuclear receptor inducer is one that induces constitutive androstane receptor (CAR) or pregnane X receptor (PXR) including but not limited to preparations from one or more of Artemisia capillaris, Artemisia scoparia, Artemisa annua, other Artemisia spp., Allium sativa, Gingko biloba, Citrus spp, Tagetes lucida, Hypericum perforatum, Piper methysticum, or Monascus purpureus.

22. A method of improving metabolism and excretion of potentially toxic compounds in animals whereby a composition as in claim 21 is administered orally, for example as a feed supplement.

23. The composition of claim 21 whereby the feed supplement is composed of one or more CAR inducers, or an extract thereof, in an amount equivalent to 0.1% to 20% of the total amount of livestock feed.

24. The composition of claim 14 whereby the nuclear receptor inducer is one that induces constitutive androstane receptor (CAR) or pregnane X receptor (PXR) including but not limited to preparations from one or more of Artemisia capillaris, Artemisia scoparia, Artemisa annua, other Artemisia spp., Allium sativa, Gingko biloba, Citrus spp, Tagetes lucida, Hypericum perforatum, Piper methysticum, or Monascus purpureus.

25. The method of increasing ovulation rate and/or litter size of a female animal whereby a composition of claim 24 is administered orally, for example as a feed supplement.

26. The composition of claim 24 whereby the feed supplement is composed of one or more CAR inducers, or an extract thereof, in an amount equivalent to 0.1% to 20% of the total mammal or livestock feed.

27. Mammal meat or livestock meat produced by a method as claimed in claim 16.

28. Livestock produced by administering said livestock with a composition comprising an active amount of one or more nuclear receptor inducers for the purpose of improving metabolism and excretion of potentially toxic compounds.

29. Livestock of claim 28, wherein the nuclear receptor inducer is one that induces constitutive androstane receptor (CAR) or pregnane X receptor (PXR) including but not limited to preparations from one or more of Artemisia capillaris, Artemisia scoparia, Artemisa annua, other Artemisia spp., Allium sativa, Gingko biloba, Citrus spp, Tagetes lucida, Hypericum perforatum, Piper methysticum, or Monascus purpureus.
